SEQUENCE LISTING (1 sequence)

SEQ ID NO: 1 - protein, 42 aa, Homo sapiens [sequence omitted]

(19) United States
(12) Patent Application Publication
(10) Pub. No.: US 2023/0027014 A1
(43) Pub. Date: Jan. 26, 2023

(54) THERAPEUTIC COMPOSITIONS COMPRISING AN AMYLOID BETA ANTIBODY OR VACCINE FOR PREVENTION AND TREATMENT OF DIASTOLIC DYSFUNCTION

(71) Applicant: AMBETEX PTY LTD, Geelong West (AU)

(72) Inventors: Sean MCGEE, Geelong West (AU); Liam HALL, Geelong West (AU); Juliane CZECZOR, Geelong West (AU)

(73) Assignee: AMBETEX PTY LTD, Geelong West (AU)

(21) Appl. No.: 17/784,585

(22) PCT Filed: Nov. 20, 2020
(86) PCT No.: PCT/AU2020/051254

§ 371 Date: Jun. 10, 2022

(30) Foreign Application Priority Data
Nov. 12, 2019 (AU) 2019904682

(51) Int. Cl. C07K 16/18; A61P 9/00

(52) U.S. Cl. CPC C07K 16/18; A61P 9/00; A61K 2039/505

(57) ABSTRACT

Methods for preventing or treating diastolic dysfunction in an individual comprising providing in an individual in need of said prevention or treatment a therapeutically effective amount of an anti-Aβ 42 antibody, compositions for providing in an individual a therapeutically effective amount of an anti-Aβ42 antibody, methods for determining likelihood of an individual having or developing diastolic dysfunction.

### FIELD OF THE INVENTION

The invention relates to diastolic dysfunction and related conditions and to antibody compositions and vaccines and therapeutic uses of same.

### BACKGROUND OF THE INVENTION

Diastole is the part of the cardiac cycle that includes the isovolumetric relaxation phase and the filling phases and has passive and active components. The filling of the left ventricle (LV) is divided into rapid filling during early diastole, diastasis, and a rapid filling phase late in diastole that corresponds with atrial contraction.

LV relaxation, an essential characteristic of normal diastole, is an energy-dependent process. In particular, adenosine triphosphate (ATP) is required to pump free myoplasmic calcium back into the sarcoplasmic reticulum, to extrude the calcium ions which enter the cell during the plateau phase of the action potential, and to extrude sodium that has been exchanged for calcium via sodium/potassium ATPase and an ATP-dependent calcium pump. Thus, when ATP production is limited, for example where there has been an impairment in the cardiac uptake of glucose and/or impairments in mitochondrial metabolism, this may result in a slower rate of isovolumic relaxation and reduced distensibility of the LV.

Left ventricular diastolic dysfunction (LVDD) is a preclinical condition defined as the inability of the LV to fill an adequate end diastolic volume (preload volume) at an acceptable pressure. LVDD is generally a consequence of abnormalities during diastole. The aforementioned impaired LV relaxation, high filling pressure, and increased LV operating stiffness are underlying mechanisms in LVDD. Cardiac impairments that represent LVDD include reduced E:A ratio and increased deceleration time. These impairments can lead to concentric hypertrophy and associated cardiomyopathy, and heart failure.

Epidemiological evidence suggests there is a latent phase in which diastolic dysfunction is present and progresses in severity before the symptoms of heart failure arise.

Asymptomatic mild LVDD is found in 21%, and moderate or severe diastolic dysfunction is present in 7% of the population.

In early diastolic dysfunction, elevated LV stiffness is associated with diastolic filling abnormalities and normal exercise tolerance. Asymptomatic diastolic dysfunction may be present for significant periods before it develops into a symptomatic clinical event. When the disease progresses, pulmonary pressures increase abnormally during exercise, producing reduced exercise tolerance. When filling pressures increase further, clinical signs of heart failure appear. In a significant number of cases of diastolic heart failure, patients have atrial fibrillation at the time of diagnosis, suggesting an association and a possible common pathogenesis. With atrial fibrillation, diastolic dysfunction may rapidly lead to overt diastolic heart failure.

The asymptomatic phase of diastolic dysfunction represents a potential time to intervene to prevent symptomatic heart failure. Suggesting the success of possible interventions, a mortality benefit has been observed in those whose diastolic dysfunction improved compared with those whose diastolic dysfunction remained the same or worsened.

Patients with LVDD are generally older, more often female, and have a high prevalence of CVD and other morbid conditions, such as obesity, metabolic syndrome, diabetes mellitus type 2, salt-sensitive hypertension, atrial fibrillation, COPD, anemia, and/or renal dysfunction.

LVDD may lead to heart failure with preserved ejection fraction (HFPeF). In HFPeF, normal ejection fraction is observed, but only at the expense of increased LV filling pressure. HFPeF is sometimes referred to as ‘diastolic heart failure’ or ‘backward heart failure’.

LVDD is an important precursor to many different cardiovascular diseases. It represents the dominant mechanism (⅔ of patients) in the development of HFPeF. HFPeF shows a rising prevalence in the older population. By 2020, more than 8% of people over 65 are estimated to have HFPEF and is associated with a poor prognosis.

To date, there are no specific treatments for diastolic dysfunction to selectively enhance myocardial relaxation. Moreover, no drug has been developed to improve long-term outcomes for diastolic heart failure.
WO2010/035261A2 discusses polypeptides, compositions, and methods of use thereof for optimized treatment of diseases or disorders associated with amyloid beta protein (Aβ) accumulation in a subject.
WO2009/052125A2 discusses a human anti-amyloid antibody, including isolated nucleic acids that encode at least one anti-amyloid antibody, amyloid, vectors, host cells, transgenic animals or plants, and methods of making and using thereof, including therapeutic compositions, methods and devices.
WO2005/028511A2 discusses an anti-amyloid antibody, including isolated nucleic acids that encode at least one anti-amyloid antibody, amyloid, vectors, host cells, transgenic animals or plants, and methods of making and using thereof, including therapeutic compositions, methods and devices.
WO2008/002893A2 discusses antibodies, including specified portions or variants, specific for at least one beta-amyloid (amyloid) protein or fragment thereof, as well as anti-idiotype antibodies, and nucleic acids encoding such anti-amyloid antibodies, complementary nucleic acids, vectors, host cells, and methods of making and using thereof, including therapeutic formulations, administration and devices.
WO2007/068412A2 discusses methods and compositions for the therapeutic and diagnostic use in the treatment of diseases and disorders which are caused by or associated with amyloid or amyloid-like proteins including amyloidosis, a group of disorders and abnormalities associated with amyloid protein such as Alzheimer's disease.
Sanna G. D et al. JACC Heart Failure, February 2019, Vol. 7, No. 2, discusses a case control study that sought to assess the presence and characteristics of cardiac abnormalities in patients with Alzheimer disease (AD).
Stamatelopoulos K et al. Rev Esp Cardio., 2017, Vol. 70, No. 11 discusses that circulating amyloid-beta (1-40) predicts clinical outcomes in patients with heart failure.
Troncone L et al. J America College of Cardiol 2016 Vol. 68, No. 22 discusses if amyloid beta (AD) protein aggregates are present in the hearts of patients with a primary diagnosis of AD, affecting myocardial function.

Tublin J. M et al., Circulation Research January 2019 Vol. 124 No. 1 discusses an overview of cardiovascular links to Alzheimer's disease.

There is a need for methods and compositions for providing improvements in the treatment or prevention of diastolic dysfunction.

### SUMMARY OF THE INVENTION

The invention relates to methods of treating, preventing, or ameliorating diastolic dysfunction or conditions associated with or arising from same, and to pharmaceutical compositions and kits for providing antibodies that bind to Aβ42 in an individual for treating or preventing diastolic dysfunction or conditions associated with or arising from same.

The invention provides a method for preventing or treating diastolic dysfunction or condition associated with same in an individual including providing in an individual a therapeutically effective amount of an anti-Aβ42 antibody.

The invention further provides a composition comprising a therapeutically effective amount of an anti-Aβ42 antibody for use in preventing or treating diastolic dysfunction or condition associated with same in an individual.

The invention further provides a use of a composition comprising an anti-Aβ42 antibody in the manufacture of a medicament for preventing or treating diastolic dysfunction or condition associated with same.

The invention further provides a method for preventing or treating diastolic dysfunction or condition associated with same in an individual comprising:
assessing or having assessed, a sample, preferably a plasma sample obtained from an individual for whom diastolic dysfunction is to be prevented or treated to determine the amount of Aβ42 in the sample; and
where the individual has an amount of Aβ42 that is greater than that observed in a control describing the amount of Aβ42 in an individual who does not develop, or does not have diastolic dysfunction;
   providing an anti-Aβ42 antibody in the individual;
      thereby preventing or treating diastolic dysfunction or condition associated with same in the individual.

The invention further provides a method for determining likelihood of an individual having or developing diastolic dysfunction comprising:
assessing or having assessed, a sample, preferably a plasma sample obtained from an individual for whom the likelihood of having or developing diastolic dysfunction is to be determined to determine the amount of Aβ42 in the sample;
determining that the individual has a higher likelihood of developing or having diastolic dysfunction where the amount of Aβ42 is greater than that observed in a control describing the amount of Aβ42 in an individual who does not develop, or does not have diastolic dysfunction;
determining that the individual has a lower likelihood of developing or having diastolic dysfunction where the amount of Aβ42 is the same as that observed in the control;
   thereby determining the likelihood of an individual having or developing diastolic dysfunction.

The invention further provides a kit comprising:
an anti-Aβ42 antibody and/or
a vaccine or immune-stimulating composition for producing an anti-Aβ42 antibody in an individual;
written instructions for use of the kit in an enumerated embodiment described below.

The invention further provides a pharmaceutical formulation comprising:
an anti-Aβ42 antibody, preferably Bapineuzumab
   wherein the antibody is provided in an amount of about 1 to 250 mg/ml of the formulation, more preferably about 10 to 200 mg/ml of the formulation, more preferably about 40 to 80 mg/ml of the formulation, preferably 80 mg/ml of the formulation, or preferably 150 to 200 mg/ml for sub-cutaneous or intramuscular administration,
   optionally wherein the formulation does not comprise a salt, or does not comprise a buffer.

Various (enumerated) embodiments of the present invention are described herein. It will be recognized that features specified in each embodiment may be combined with other specified features to provide further embodiments of the present disclosure.

Embodiment 1: A method for preventing or treating diastolic dysfunction in an individual, preferably an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 2: A method for preventing or treating heart failure, more preferably HFpEF in an individual, preferably an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 3: A method for preventing or treating concentric hypertrophy in an individual, preferably an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 4: A method for preserving or decreasing left ventricle deceleration time in an individual, preferably in an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 5: A method for preserving or preventing intra-ventricular septal thickening in an individual, preferably in an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 6: A method for preserving or preventing an increase in left ventricular mass in an individual, preferably in an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 7: A method for preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 8: A method for preventing the reduction of cardiac glucose uptake, or for preventing the accumulation of cardiac tri-acyl glycerol in an individual, preferably an obese, or pre-diabetic, or diabetic or elderly individual, more preferably an obese individual, or an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 9: A method for preventing or treating obesity-associated cardiomyopathy in an individual, more preferably in an individual having an elevated amount of Aβ42, more preferably an elevated amount of plasma Aβ42, comprising administering a therapeutically effective amount of an anti-Aβ42 antibody, more preferably Bapineuzumab to the individual, preferably in an amount of about 0.1 mg/kg to 15 mg/kg, preferably once every 14 days.

Embodiment 10: A method for preventing or treating diastolic dysfunction in an individual, preferably in an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 11: A method for preventing or treating heart failure, more preferably HFpEF in an individual, preferably in an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 12: A method for preventing or treating concentric hypertrophy in an individual, preferably an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 13: A method for preserving or decreasing left ventricle deceleration time in an individual, preferably an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 14: A method for preserving or preventing intra-ventricular septal thickening in an individual, preferably an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 15: A method for preserving or preventing an increase in left ventricular mass in an individual, preferably in an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 16: A method for preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 17: A method for preventing the reduction of cardiac glucose uptake, or for preventing the accumulation of cardiac tri-acyl glycerol in an individual, preferably an obese, or pre-diabetic, or diabetic or elderly individual, more preferably an obese individual, or an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 18: A method for preventing or treating obesity-associated cardiomyopathy in an individual, more preferably in an individual having an elevated amount of Aβ42, more preferably an elevated amount of plasma Aβ42, comprising administering a vaccine or immune-stimulating composition to the individual to produce in the individual a therapeutically effective amount of an anti-Aβ42 antibody.

Embodiment 19: A composition for use in preventing or treating diastolic dysfunction in an individual, preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 20: A composition for use in preventing or treating heart failure, more preferably HFpEF in an individual, preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 21: A composition for use preventing or treating concentric hypertrophy in an individual, preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 22: A composition for use in preserving or decreasing left ventricle deceleration time in an individual in an individual, preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 23: A composition for use in preserving or preventing intra-ventricular septal thickening in an individual preferably an obese, or pre-diabetic, or diabetic or elderly individual, more preferably an obese individual or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 24: A composition for use in preserving or preventing an increase in left ventricular mass in an individual preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to preferably in an amount of about 0.1 mg/kg to 15 mg/kg, preferably once every 14 days.

Embodiment 25: A composition for use preventing or treating cardiomyopathy, more preferably diabetic cardiomyopathy, or hypertrophic cardiomyopathy, or ischemic cardiomyopathy, or hypertensive cardiomyopathy in an individual preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, preferably an anti-Aβ42 antibody, more preferably Bapineuzumab to the individual, preferably in an amount of about 0.1 mg/kg to 15 mg/kg, preferably once every 14 days.

Embodiment 26: A composition for use in preventing the reduction of cardiac glucose uptake, or for preventing the accumulation of cardiac tri-acyl glycerol in an individual preferably an obese, or pre-diabetic, or diabetic or elderly individual, more preferably an obese individual, or an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 27: A composition for use in preventing or treating obesity-associated cardiomyopathy in an individual, more preferably in an individual having an elevated amount of Aβ42, more preferably an elevated amount of plasma Aβ42, comprising a therapeutically effective amount of an anti-Aβ42 antibody, more preferably Bapineuzumab to the individual, preferably in an amount of about 0.1 mg/kg to 15 mg/kg, preferably once every 14 days.

Embodiment 28: A composition for use in preventing or treating diastolic dysfunction in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 29: A composition for use in preventing or treating heart failure, more preferably HFpEF, in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 30: A composition for use in preventing or treating concentric hypertrophy in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 31: A composition for use in preserving or decreasing left ventricle deceleration time in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 32: A composition for use in preserving or preventing intra-ventricular septal thickening in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 33: A composition for use in in preserving or preventing an increase in left ventricular mass in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 34: A composition for use in preventing or treating cardiomyopathy, more preferably diabetic cardiomyopathy, or hypertrophic cardiomyopathy, or ischemic cardiomyopathy, or hypertensive cardiomyopathy, in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of anti-Aβ42 antibody.

Embodiment 35: A composition for use in preventing the reduction of cardiac glucose uptake, or for preventing the accumulation of cardiac tri-acyl glycerol in an individual, preferably an obese, or pre-diabetic, or diabetic or elderly individual, more preferably an obese individual, or an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 36: A composition for use in preventing or treating obesity-associated cardiomyopathy in an individual, more preferably in an individual having an elevated amount of Aβ42, more preferably an elevated amount of plasma Aβ42, comprising a vaccine or immune-stimulating composition for producing in the individual a therapeutically effective amount of an anti-Aβ42 antibody.

Embodiment 37: A pharmaceutical formulation, preferably a formulation for sub-cutaneous injection, the formulation comprising:
an anti-Aβ42 antibody, preferably Bapineuzumab
a surfactant
a polyol
   wherein the antibody is provided in an amount of about 1 to 250 mg/ml of the formulation, more preferably about 10 to 200 mg/ml of the formulation, more preferably about 40 to 80 mg/ml of the formulation, preferably 80 mg/ml of the formulation, or from 50 to 200 mg/ml of the formulation for sub cutaneous or intra muscular administration,
   optionally wherein the formulation does not comprise a salt, or does not comprise a buffer.

Embodiment 38: A method for determining the likelihood of an individual having or developing diastolic dysfunction comprising:
assessing or having assessed a sample, preferably a plasma sample, obtained from an individual for whom the likelihood of having or developing diastolic dysfunction is to be determined, to determine the amount of Aβ42 in the sample;
determining that the individual has a higher likelihood of developing or having diastolic dysfunction where the amount of Aβ42 is greater than that observed in a control describing the amount of Aβ42 in an individual who does not develop, or does not have diastolic dysfunction;
determining that the individual has a lower likelihood of developing or having diastolic dysfunction where the amount of Aβ42 is the same as that observed in the control;
   preferably wherein the individual is overweight or obese, preferably wherein the control describes an amount of Aβ42 in an individual who does not develop, or does not have diastolic dysfunction and who is not elderly, preferably of an age of from 20 to 40 years old, and having a body mass index in the normal range,
   thereby determining the likelihood of an individual having or developing diastolic dysfunction. In any one of the above enumerated embodiments, the individual may be assessed for an amount of Aβ42, preferably plasma Aβ42, prior to administration or use of the composition according to the relevant embodiment.

### DETAILED DESCRIPTION OF THE INVENTION

The isovolumetric relaxation phase is an essential phase of normal diastole. It is energy dependent, and aberrations of the relaxation phase, as observed in LVDD and related clinical manifestations such as concentric hypertrophy and later heart failure, occur where there is an impairment in availability of ATP, for example as occurring where there is reduced cardiac glucose uptake.

It has been found herein that chronic exposure to Aβ42 results in impairments in cardiac metabolism, including a reduction in cardiac glucose uptake, accumulation in cardiac TAG and impairment in cardiac function including concentric hypertrophy, and that these outcomes are minimised by providing anti-Aβ42 antibodies in individuals, particularly those having a high fat content diet and/or whom are overweight or obese.

Without wanting to be bound by hypothesis it is believed that chronic exposure to Aβ42 causes or otherwise results in cardiomyocyte inflammation leading to impaired cardiomyocyte metabolism, reducing their glucose uptake, shunting of glucose into TAG and TAG accumulation, and that antibody administration either reduces or neutralises plasma Aβ42 thereby minimising these pathological outcomes.

### 1. DEFINITIONS

For purposes of interpreting this specification, the following definitions will apply and whenever appropriate, terms used in the singular will also include the plural and vice versa.

Aβ used herein, the term “about” in relation to a numerical value×means+/−10%, unless the context dictates otherwise.

As used herein, the term “Amyloid beta” (Aβ or Abeta) denotes peptides of 36-43 amino acids, preferably Aβ42 that are crucially involved in Alzheimer's disease as the main component of the amyloid plaques found in the brains of Alzheimer patients. The peptides derive from the amyloid precursor protein (APP), which is cleaved by beta secretase and gamma secretase to yield Aβ. Aβ molecules can aggregate to form flexible soluble oligomers which may exist in several forms.

Aβ used herein, the term “anti-amyloid beta antibody” or “anti-Aβ antibody” or “anti-Abeta antibody” refers to an immunoglobulin molecule or fragment thereof, such as a CDR, variable domain or fragment thereof, Fab, Dab fragment or whole antibody. An anti-Aβ antibody may be of any isotype or subtype and may be xenogeneic, allogeneic or syngeneic. An anti-Aβ antibody may bind to any Aβ protein or peptide or molecule comprising same such as APP. An anti-Aβ antibody may bind to Aβ or a fragment thereof in soluble form (i.e. when partially or wholly soluble in plasma) or in insoluble form, for example when presented as a plaque. An anti-Aβ antibody may bind to Aβ and thereby enable depletion of Aβ from plasma and excretion from the body; or may enable neutralisation of Aβ, for example thereby minimising a toxic effect of Aβ such as cardiomyocyte inflammation.

Aβ used herein, the term “diastolic dysfunction” generally refers to a condition characterised by the inability of the left ventricle to fill an adequate end diastolic volume at a physiologically normal or acceptable pressure.

Aβ used herein, the term “E/A ratio” generally refers to the ratio of the E wave to the A wave. On echocardiography, the peak velocity of blood flow across the mitral valve during early diastolic filling corresponds to the E wave. Similarly, atrial contraction corresponds to the A wave. From these findings, “the E/A ratio” is calculated. Under normal conditions, E is greater than A and the E/A ratio is approximately 1.5. In early diastolic dysfunction, relaxation is impaired and, with vigorous atrial contraction, the E/A ratio decreases to less than 1.0. Aβ the disease progresses, left ventricular compliance is reduced, which increases left atrial pressure and, in turn, increases early left ventricular filling despite impaired relaxation. This paradoxical normalization of the E/A ratio may be called “pseudonormalization”. In patients with severe diastolic dysfunction, left ventricular filling occurs primarily in early diastole, creating an E/A ratio greater than 2.0.

Aβ used herein, “deceleration time” is the time taken from the maximum E point to baseline. In adults, it is normally less than 220 milliseconds.

Aβ used herein, the term “concentric hypertrophy” generally refers to a form of cardiac hypertrophy associated with increased left ventricular wall thickness, or associated with an increase in LV mass without dilation of the LV, for example as measured by LVIDd. An increase in pressure, common in hypertension or resistance training, results in a concentric hypertrophic phenotype. Concentric hypertrophy differs from “eccentric hypertrophy”, the latter being characterised by dilatation of the left ventricular chamber and is observed in, or associated with valvular defects or endurance training.

Eccentric hypertrophy may develop from concentric hypertrophy.

An individual with diastolic dysfunction, in particular, an individual with early stage diastolic dysfunction may or may not have detectable concentric hypertrophy.

Aβ used herein, the term “HFpEF” or “heart failure with preserved ejection fraction” generally refers to a form of heart failure characterised by normal ejection fraction (at or above about 50% of ventricle volume) dependent on increased LV pressure.

Aβ used herein, “Cardiomyopathy” generally refers to a heterogeneous group of diseases of the myocardium associated with mechanical and/or electrical dysfunction, which usually (but not invariably) exhibit inappropriate ventricular hypertrophy or dilatation. Cardiomyopathy may be a primary cardiomyopathy, which is confined to the heart, preferably an acquired cardiomyopathy, more preferably an obesity-associated cardiomyopathy. An obesity-associated cardiomyopathy is defined myocardial disease in obese individuals that cannot be explained by diabetes mellitus, hypertension, coronary artery disease or other etiologies. The presentation of this disease can vary from asymptomatic left ventricular dysfunction to overt dilated cardiomyopathy and heart failure.

Aβ used herein, the term “elderly individual” refers to an individual over 60 years of age, more preferably 65 or 70 or 75 years of age.

Aβ used herein, the term “pharmaceutically acceptable” means a nontoxic material that does not interfere with the effectiveness of the biological activity of the active ingredient(s).

Aβ used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, {e.g., stabilization of a discernible symptom), physiologically, {e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

Aβ used herein, the term “therapeutically effective amount” refers to an amount of the compound of the invention, e.g. anti-Aβ42 antibody; or vaccine or immunostimulating composition for producing same; which is sufficient to achieve the stated effect. Accordingly, a therapeutically effective amount of an anti-Aβ42 antibody; or vaccine or immunostimulating composition for producing same; will be an amount sufficient for the treatment or prevention of the condition mediated by or associated with Aβ42 plasma expression or production.

By “therapeutic regimen” is meant the pattern of treatment of an illness, e.g., the pattern of dosing used during the treatment of the disease or disorder.

Aβ used herein, a subject is “in need of” a treatment if such subject would benefit biologically, medically or in quality of life from such treatment.

### 2. DETAILED DESCRIPTION OF THE FIGURES

FIG. 1—Chronic Aβ42 administration alters cardiac metabolism.

FIG. 2—Chronic Aβ42 administration alters cardiac function.

FIG. 3—Administration of anti-Aβ42 antibodies preserves diastolic function in development of obesity.

FIG. 4—Administration of anti-Aβ42 antibodies prevents concentric hypertrophy in development of obesity.

FIG. 5—Administration of anti-Aβ42 antibodies preserves diastolic function in established obesity.

FIG. 6—Chronic Aβ40 administration does not alter cardiac function.

### 3. MODES OF CARRYING OUT THE INVENTION

3.1 Individuals

An individual to whom the methods of the invention are applied is mammalian, preferably a human being.

An individual may not have diastolic dysfunction at the time of treatment. Such an individual may be at risk for diastolic dysfunction i.e. may have one or more risk factors for diastolic dysfunction. For example, the individual may be pre diabetic or diabetic, overweight or obese, female, have Alzheimer's disease or other neural disease with Aβ involvement, or elderly. The individual may have an elevated amount of Aβ42, preferably an elevated amount of plasma Aβ42. The invention may be applied to such an individual to prevent the development of diastolic dysfunction, or to prevent diastolic dysfunction.

In another embodiment, an individual may have diastolic dysfunction at the time of treatment. Such an individual may be asymptomatic for diastolic dysfunction, or symptomatic for diastolic dysfunction. The invention may be applied to such an individual to treat or ameliorate or alleviate diastolic dysfunction.

In one embodiment, the individual to be administered an anti-Aβ42 antibody or immune-stimulation composition is obese and has an elevated amount of plasma Aβ42 and may or may not have diastolic dysfunction. Such an individual may have obesity associated cardiomyopathy, or may be at risk for same.

Stages of diastolic dysfunction have been classified according to various grading systems. For example, four basic echocardiographic patterns of diastolic dysfunction, (graded I to IV) according to the American Society of Echocardiography and the European Association of Cardiovascular Imaging are described:
Grade I diastolic dysfunction. On the mitral inflow Doppler echocardiogram, the E/A ratio is ≤0.8 and deceleration time is >200 ms, while the E/e′ ratio, a measure of the filling pressure, is within normal limits at <10. This pattern may develop normally with age in some patients, and many grade I patients will not have any clinical signs or symptoms of heart failure.
Grade II diastolic dysfunction is called “pseudonormal filling dynamics”, with the E/A ratio between 0.8 and 2.0, and a reduction in deceleration time to between 160 and 220 ms. This is considered moderate diastolic dysfunction and is associated with elevated left atrial filling pressures, with an E/e′ ratio between 10 and 14. These patients more commonly have symptoms of heart failure, and many have left atrial enlargement due to the elevated pressures in the left heart.
Class III diastolic dysfunction patients have an E/A ratio >2 and E/e′ ratio >14. They will demonstrate reversal of their diastolic abnormalities on echocardiogram when they perform the Valsalva maneuver. This is referred to as “reversible restrictive diastolic dysfunction”.
Class IV diastolic dysfunction patients will not demonstrate reversibility of their echocardiogram abnormalities, and are therefore said to suffer from “fixed restrictive diastolic dysfunction”.

Grade III and IV diastolic dysfunction are called “restrictive filling dynamics”. These are both severe forms of diastolic dysfunction, and patients tend to have advanced heart failure symptoms.

In one embodiment, an individual having Grade I diastolic dysfunction (as described above), preferably having an elevated plasma amount of Aβ42 is provided with anti-Aβ42 antibodies to prevent the development of more severe diastolic dysfuction, or otherwise to preserve diastolic function.

In one embodiment, an individual having Grade II, III or IV diastolic dysfunction (as described above), preferably having an elevated plasma amount of Aβ42 is provided with anti-Aβ42 antibodies to treat or reverse diastolic dysfuction, or to treat or reverse one or more symptoms or characters of diastolic dysfunction.

In one embodiment, an individual may have concentric hypertrophy.

An individual in need of treatment may have a normal left ventricle diameter and may have a normal cardiac weight.

An individual in need of treatment may have an increased LV deceleration time.

An individual in need of treatment may have a cardiomyopathy, especially an ischemic or hypertrophic cardiomyopathy.

An individual in need of treatment may have a systolic condition in addition to diastolic dysfunction.

An individual the subject of treatment may be symptomatic for heart failure and may be symptomatic for HFPpEF or may be asymptomatic for heart failure or HFpEF. Symptoms of heart failure generally include shortness of breath including exercise induced dyspnea, paroxysmal nocturnal dyspnea and orthopnea, exercise intolerance, fatigue, elevated jugular venous pressure, and edema. Patients with HFpEF poorly tolerate stress, particularly hemodynamic alterations of ventricular loading or increased diastolic pressures. Often there is a more dramatic elevation in systolic blood pressure in HFpEF.

An individual who is asymptomatic or symptomatic for heart failure may or may not be obese or overweight, diabetic or pre-diabetic, have Alzheimer's disease or other neural disease with Aβ involvement, or elderly.

### 3.2 Screening Individuals for LVDD

In a particularly preferred embodiment, an individual may be selected for treatment or prevention of LVDD, or screened for LVDD, or assessed for risk of developing LVDD by assessing or measuring the plasma amount of Aβ and optionally comparing with a normal control describing an amount of Aβ in plasma in an individual not having, or not at risk of having diastolic dysfunction, for example, an individual who is not overweight or obese, or not pre-diabetic or diabetic, or who does not have Alzheimer's disease or who is not elderly.

In one embodiment, a control may be an age matched control. Where the individual to be assessed is elderly, the control may describe an amount of Aβ42 in plasma that is consistent with that found in a normal individual having an age of about 20 to 40 years old.

In one embodiment a control describes the amount of Aβ42 in plasma from an individual having a body mass index in the normal range, from about 18.5 to 24.9 kg/m².

In one embodiment, a control describing the amount of Aβ42 in plasma may be may be derived from a single individual. In another embodiment, a control may be derived from a cohort of individuals.

It has been established in the Examples herein that diastolic dysfunction is induced by administration of an amount of about 0.04 mg/kg of Aβ42 peptide per day. Further, individuals on a high fat diet may develop a plasma amount of Aβ42 peptide of about 3 fold above controls. In one embodiment, an individual to be selected for treatment may have a plasma amount of Aβ42 peptide of about 10 to 100 pM, or about 1 to at least 10 fold the amount of Aβ42 peptide in a control.

A control may provide a reference point against which a determination regarding implementation of subsequent prophylaxis or therapy can be made. The determination may be made on the basis of the comparison between test sample obtained from the individual being assessed for prophylaxis or treatment and the control.

In certain embodiments, the control may be provided in the form of data that has been derived by another party, and/or prior to assessment of the subject for treatment. For example, the control may be derived from a commercial database or a publically available database.

In one embodiment the individual is selected for treatment or prevention of LVDD, or screened for LVDD, or assessed for risk of developing LVDD, where the individual has an amount of Aβ or fragment thereof, preferably Aβ42 that is greater than the amount of Aβ or fragment thereof, preferably Aβ42 in a normal control.

Methods for measurement of plasma amounts of Aβ or fragment thereof such as Aβ42 are known in the art: [Kim et al., Sci. Adv. 2019; 5:eaav1388 17 Apr. 2019; Shie, F S et al., PLOSONE|DOI:10.1371/journal.pone.0134531 Aug. 5, 2015; Balakrishnan K et al. Journal of Alzheimer's Disease 8 (2005) 269-282; Luciano R et al., PEDIATRICS Volume 135, number 6, June 2015].

In certain embodiments, the samples to be tested are body fluids such as blood, serum, plasma, urine, tears, saliva, CSF and the like.

In certain embodiments, the sample from the individual may require processing prior to detection of the levels of Aβ42. For example, the sample may be centrifuged or diluted to a particular concentration or adjusted to a particular pH prior to testing.

Conversely, it may be desirable to concentrate a sample that is too dilute, prior to testing.

In certain embodiments Aβ42 may be measured, or peptides or complexes that comprise Aβ42 may be measured.

In other embodiments, fragments of Aβ42 comprising the Aβ42 C-terminal sequences that distinguish Aβ42 from Aβ40 may be measured.

The above described methods may be combined with the following diagnostic procedures for detecting, assessing or measuring diastolic dysfunction or related heart failure such as HFPeF, or the following procedures may be used without assessment of plasma amount of Aβ42.

Two-dimensional echocardiography with Doppler flow measurements is commonly used to assess diastolic dysfunction. Exercise may be required to clearly demonstrate diastolic functional changes.

During diastole, blood flows through the mitral valve when the LV relaxes, causing an early diastolic mitral velocity (E), and then additional blood is pumped through the valve when the left atrium contracts during late diastole (A). The E/A ratio can be altered in diastolic dysfunction.

Tissue Doppler imaging is an echocardiographic technique that measures the velocity of the mitral annulus. This velocity has been shown to be a sensitive marker of early myocardial dysfunction. With abnormal active relaxation, mitral annulus velocity during early diastole (E) is decreased while mitral annulus velocity during late diastole (A) is increased, resulting in a lowered E/A ratio. In animal models, tissue Doppler imaging has been validated as a reliable tool for the evaluation of diastolic dysfunction.

The E- and A-wave velocities are affected by blood volume, mitral valve anatomy, mitral valve function, and atrial fibrillation, making standard echocardiography less reliable. In these cases, tissue Doppler imaging is useful for measuring mitral annular motion (a measure of transmitral flow that is independent of the aforementioned factors). Cardiac catheterization remains the preferred method for diagnosing diastolic dysfunction. However, in day-to-day clinical practice, two-dimensional echocardiography with Doppler is the best noninvasive tool to confirm the diagnosis. Rarely, radionuclide angiography is used for patients in whom echocardiography is technically difficult. LV inflow propagation velocity (VP) by color M-mode Doppler is another relatively preload-insensitive index of LV relaxation.

It has been shown to correlate well with the time constant of isovolumic relaxation (W), both in animals and humans.

Recently, speckle tracking echocardiography (STE) has emerged as a promising technique for the evaluation of myocardial wall motion by strain analysis. By tracking the displacement of speckles during the cardiac cycle, STE allows semiautomated delineation of myocardial deformation.

Cardiac magnetic resonance (CMR) imaging is a newer technique for measuring diastolic dysfunction. Myocardial tagging allows the labeling of specific myocardial regions. Following these regions during diastole enables them to be analyzed in a manner similar to STE. In addition, the rapid diastolic untwisting motion followed by CMR tagging is directly related to isovolumic relaxation and can be used as an index of the rate and completeness of relaxation.

Biomarkers may also be assessed for diagnosis of LVDD. B-type natriuretic peptide (BNP) and TnI have been used as HF biomarkers and exhibit strong association with hospitalization.

Nevertheless, they are nonspecific and not well correlated with diastolic dysfunction. Recently, it has been reported that cMyBP-C could be a new biomarker releases from damaged myofilaments. Additionally, elevated S-glutathionylated cMyBP-C level can be detected in the blood of patients with diastolic dysfunction. Hypertension and diabetes lead to cardiac oxidation and S-glutathionylation of cMyBP-C, a cardiac contractile protein, which leads to impaired relaxation, and modified cMyBP-C in the blood may represent a circulating biomarker for diastolic dysfunction.

### 3.3 Anti-Aβ Antibodies

Anti-AS antibodies for use in the invention generally bind to plasma or extracellular associated Aβ42, especially oligomeric Aβ42. Without wanting to be bound by hypothesis, it is believed that the administration of anti-Aβ antibodies either depletes, binds or neutralises plasma or extracellular associated Aβ42 peptide, resulting in a minimisation of diastolic dysfunction, preferably through a minimisation of Aβ42 induced or associated cardiomyocyte inflammation and/or reduced cardiac glucose uptake.

The anti-Aβ antibodies may bind to any one of the following epitopes on Aβ42 peptide shown in Table 1:

TABLE 1
Epitope*
AA 1-5
AA 16-26
AA 3-12
AA 18-27
AA 13-24
AA 30-40
AA 3-6
*Numbering with reference to the amino acid sequence of A342

(DAEFRHDSGYEVHHQKLVFFAEDVGSNKGAIIGLMVGGVVIA)

The anti-Aβ antibodies may bind to monomeric Aβ42 or to oligomeric Aβ42 peptide.

The anti-Aβ antibodies may bind to an amyloid fibril.

The anti-AP antibodies may bind to an amyloid protofibril.

“Selective anti-Aβ42 antibodies”, i.e. antibodies that bind to Aβ42 but not to Aβ40; and “selective anti-Aβ40 antibodies”, i.e. antibodies that bind to Aβ40 but not to Aβ42, and their methods of synthesis are known in the art. See for example: Ida N. et al. J. Biol. Chem. 1996 271: 22908-22914; Axelsen T V et al. Mol. Immunol. 2009; 46: 2267-2273; Miller D L, et al J. Alzheimers Dis. 2011; 23: 293-305.

In one embodiment an anti-Aβ antibody may be a selective anti Aβ42 antibody.

The anti-Aβ antibodies may be human, humanized, chimeric, murine or derived from another mammalian species, or avian.

The anti-Aβ antibodies may be of any isotype or subtype. For example the anti-Aβ antibodies may be IgG, IgM, IgA, IgE or IgD.

Preferably the antibody is IgG, more preferably of subtype IgG1, IgG2 or IgG4.

The anti-Aβ antibodies may be whole antibodies, i.e comprising all of the domains common to the relevant isotype or subtype, or the anti-Aβ antibodies may be a fragment of a whole antibody comprising at least CDR and framework regions for enabling binding of the anti-Aβ antibodies to an epitope on Aβ peptide.

The anti-Aβ antibodies may be in the form of a Fab, Dab.

The anti-Aβ antibodies may be selected from the group consisting of Bapineuzumab, Solanezumab, Gantenerumab, Crenezumab, Aducanumab, BAN2401 and MEDI1814. These anti-Aβ antibodies and the manufacture thereof are described in the patent specifications referred to in Table 2. The entire contents of the patent specifications referred to in Table 2 are incorporated herein by reference.

TABLE 2

| anti-Aβ antibody | Reference |
|---|---|
| Bapineuzumab | WO2009/017467 |
| Solanezumab | W02001/062801 |
| Gantenerumab | WO2003/070760 |
| Crenezumab | WO2007/068412 |
| Ponezumab | WO2004/032868 |
| Aducanumab | WO2008/081008 |
| BAN2401 | WO2005/123775 |
| MEDI1814 | WO/2014/060444 |

Bapineuzumab (AAB-001; Pfizer Inc., New York, N.Y., and Janssen Pharmaceuticals, Inc., Raritan, N.J.), is a humanized immunoglobulin (Ig) G1 anti-Aβ mAb, that binds the five N-terminal residues and clears both fibrillar and soluble Aβ.

Solanezumab (LY2062430; Eli Lilly and Company, Indianapolis, Ind.), is a humanized IgG1 mAb that binds the mid-domain of Aβ (residues 16-26) and increases clearance of monomers.

Gantenerumab (Hoffman-La Roche, Basel, Switzerland), the first fully human IgG1 anti-Aβ mAb, binds a conformational epitope expressed on Aβ fibrils. This epitope encompasses both N-terminal (3-12) and central (18-27) amino acids of Aβ and thus requires that the peptide be folded with the midregion near the N-terminus.

Crenezumab (MABT5102A; Genentech, Inc., South San Francisco, Calif.) is an antibody engineered on an IgG4 backbone to minimize the activation of Fc gamma receptors. Crenezumab prefers the mid-domain of the Aβ peptide (residues 13-24) and binds multiple conformations of Aβ (monomers, oligomers, fibrils), with a 10-fold higher affinity for oligomers versus monomers. The epitope recognized by crenezumab overlaps that of solanezumab, explaining their observed crossreactivity but not their different binding profiles for various species of Aβ. It has been reported that crenezumab and solanezumab actually target slightly different epitopes (residues 13-24 vs. 16-26, respectively) and suggested that solanezumab-bound Aβ possesses an alpha-helical structure between residues 21 and 26, whereas crenezumab-bound Aβ has a random coil structure between residues 21 and 24. It has been proposed that the alpha-helical epitope is present in monomeric Aβ but absent from aggregated species, potentially explaining solanezumab's preference for monomers but crenezumab's recognition of multiple species, including oligomers.

Aducanumab (BIIB037; Biogen, Inc., Cambridge, Mass.) is a fully human IgG1 mAb that selectively reacts with Aβ aggregates, including soluble oligomers and insoluble fibrils. It binds the N-terminus (residues 3-6) and recognizes a conformational epitope present on aggregated species of Aβ but absent from monomers.

BAN2401 (BioArctic Neuroscience AB, Stockholm, Sweden, and Eisai Co., Ltd., Tokyo, Japan) is a humanized IgG1 mAb that selectively binds and clears soluble Aβ protofibrils. It was derived from the E22G Arctic mutation in the amyloid precursor protein.

MEDI1814 (MedImmune, UK and Astra Zeneca UK) is a fully human IgGλ monoclonal antibody engineered for selective, high-affinity binding of Aβ x-42 (Aβ42) peptides as well as to have a greatly reduced effector function by introducing a triple mutation into the Fc region. In vitro, MEDI1814 binds specifically all forms of Aβ42 peptides but not Aβ40 peptides. In rats and cynomolgous monkeys, MEDI1814 increases total and decreases free CSFAβ42 levels, but does not affect total CSF Aβ40 levels.

An anti-Aβ antibody may be provided in the form of a pharmaceutical composition including a therapeutically effective amount of an anti-Aβ antibody and a pharmaceutically acceptable carrier. A pharmaceutical composition may be in solid or liquid form and may be, inter alia, in a form of powder, tablet, solution or aerosol.

It is preferred that a pharmaceutical composition comprises a pharmaceutically acceptable carrier and/or diluent. Examples of suitable pharmaceutical carriers, excipients and/or diluents are well known in the art and include phosphate buffered saline solutions, water, emulsions, such as oil/water emulsions, various types of wetting agents, sterile solutions etc.

Compositions comprising such carriers can be formulated by well known conventional methods.

Preparations for parenteral administration include sterile aqueous or non-aqueous solutions, suspensions, and emulsions.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils.

Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

3.4 Antibody administration

An anti-Aβ antibody may be administered by any known route enabling systemic accumulation of a therapeutically effective amount of antibody. Preferably the antibody is administered parenterally. It is particularly preferred that the administration is carried out by injection for example by intravenous (i.v.), subcutaneous (s.c), intraperitoneal (i.p.), intramuscular (i.m.), intradermal (i.d.) injection, or by intranasal or intrabronchial delivery. Other parenteral routes of administration of therapeutic antibody are known to the skilled worker.

The dosage regimen will be determined by the attending physician and clinical factors. Aβ is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 15 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute. Progress can be monitored by periodic assessment. The compositions of the invention may be administered locally or systemically.

In one embodiment the antibody is administered in an amount of 0.1 to 15 mg/kg, preferably 0.5 mg/kg to 10 mg/kg, preferably about 5 mg/kg. In these embodiments, the antibody may be administered i.v. In these embodiments, the antibody may be administered once every 2 to 4 weeks.

In one embodiment the antibody is administered in an amount of 0.5 to 5 mg/kg by i.v. administration every 10 to 15 weeks.

In one embodiment the antibody is administered in an amount of 100 to 500 mg, preferably about 400 mg (irrespective of body weight) by i.v. administration every 4 weeks.

In one embodiment the antibody is administered in an amount of from 100 to 300 mg by s.c. injection once every 4 weeks.

In one embodiment the administration of the antibody enables the reduction of the amount or activity of Aβ42 in plasma to an amount that is the same as, or that approximates that observed in a normal individual, as described above.

3.5 anti-Aβ42 formulations Anti-Aβ42 antibodies may be provided in the form of high protein content formulations in which the antibody component is from 1 to 250 mg/mL, preferably 10 to 100 mg/ml of the formulation, more preferably about 40 to 80 mg/ml of the formulation, preferably 80 mg/ml of the formulation, or preferably 150 to 200 mg/ml for sub-cutaneous or intramuscular administration. High antibody content formulations are particularly useful for management or for prevention or treatment of diastolic dysfunction, as they enable larger doses of antibody, and smaller volumes of the formulation to be administered. This minimises the pain incurred during self parenteral administration, in particular administration by self sub-cutaneous injection.

In one embodiment, the anti-Aβ42 formulations of the invention are adapted to have improved stability and solubility, minimised aggregation, and minimised viscosity at high protein content of antibody. These improvements may arise from the incorporation of a surfactant/and or a polyol.

A surfactant may be selected from the group consisting of nonionic surfactants such as polysorbates or poloxamers. In one embodiment, a surfactant may be a poloxamer, e.g., Poloxamer 188, Poloxamer 407; polyoxyethylene alkyl ethers, e.g., Brij 35, Cremophor A25, Sympatens ALM/230. A polysorbate may be Polysorbate 20 (Tween 20), Polysorbate 80 (Tween 80), Mirj, and Poloxamers, e.g., Poloxamer 188.

Surfactants may be included in an amount of about 0.1-1.5 mg/ml of the formulation.

A polyol may be selected from the group consisting of fructose, mannose, maltose, lactose, arabinose, xylose, ribose, rhamnose, galactose, glucose, sucrose, trehalose, sorbose, melezitose, raffinose, mannitol, xylitol, erythritol, threitol, sorbitol, glycerol, L-gluconate and metallic salts thereof. Preferably the polyol is sorbitol or mannitol, more preferably mannitol.

Polyols may be included in an amount of about 1 to 50 mg/mL of the formulation.

In one embodiment, the formulation does not require the use of a buffer such as acetate, succinate, gluconate, histidine, methionine, citrate, phosphate, citrate/phosphate, imidazole, combinations thereof, and other organic acid buffers.

In a particularly preferred embodiment there is provided an injectable aqueous pharmaceutical formulation comprising
an anti-Aβ42 antibody, preferably Bapineuzemab,
   wherein the antibody is provided in an amount of about 1 to 250 mg/mL, preferably 150 to 250 mg/ml of the formulation, more preferably about 200 mg/ml of the formulation;
a surfactant, preferably polysorbate 80 in an amount of about 0.1-1.5 mg/ml of the formulation;
a polyol, preferably mannitol in an amount of about 1 to 50 mg/mL of the formulation
   wherein the formulation does not contain a buffer selected from the group consisting of acetate, succinate, gluconate, histidine, methionine, citrate, phosphate, citrate/phosphate, imidazole, combinations thereof, and other organic acid buffers.

### 3.6 Aβ Immuno-Stimulating Compositions

Anti-Aβ antibodies may be provided in an individual for prevention, treatment or amelioration of diastolic dysfunction or related condition by administering an Aβ immuno-stimulating compositions or vaccine to the individual.

A vaccine or immune-stimulating composition for producing anti-Aβ antibodies may comprise a peptide immunogen as an immunogenic component for production of anti-Aβ antibodies in the individual, generally antibodies that can bind to oligomeric Aβ42.

A peptide immunogen for producing anti-Aβ42 antibodies may be synthetic or may be derived from a natural source.

A peptide immunogen for producing anti-A42 antibodies may form an AP epitope. The epitope may be as shown in Table 1, or as discussed in Ida supra; Axelsen supra; Miller supra.

These peptide immunogens, immune-stimulating compositions or vaccines comprising same are described in the patent specifications or other documents referred to in Table 3. The entire contents of the patent specifications or other documents referred to in Table 3 are incorporated herein by reference.

TABLE 3

| Peptide immunogen for | |
|---|---|
| producing anti-Aβ42 | |
| antibodies | Reference |
| AN1792, AIP-001 | WO2000072880 |
| (Elan/Wyeth) | |
| CAD106 (Novartis) | WO2004/016282 |
| ACC-001 (Pfizer) | Hagen M, Seubert P, Jacobsen S, Schenk |
| | D, Pride M, Arumugham R, Warner G, |
| | Kinney G. The Aβ peptide conjugate |
| | vaccine, ACC-001, generates N-terminal |
| | anti-Aβ antibodies in the absence of Aβ |
| | directed T-cell responses [abstract] |
| | Alzheimers Dement. 2011;6:S460-S461. |
| Affitope AD02 | WO2006/005707 |
| (Affiriis AG) | |
| ABvac 40 (Araclon | La Costa A.M et al. Safety, tolerability |
| Biotech) | and immunogenicity of an active anti- |
| | Aβ₄₀ vaccine (ABvac40) in patients with |
| | Alzheimer's disease: a randomised, |
| | double-blind, placebo-controlled, phase |
| | I trial Alzheimer's Research & |
| | Therapyvolume 10, Article number: 12 |
| | (2018) |
| ACI24 (AC Immune SA) | WO2005/081872 |
| Lu AF20513 | Davtyan H et al. Immunogenicity, Efficacy, |
| (Lundbeck/Otsuka) | Safety, and Mechanism of Action of Epitope |
| | Vaccine (Lu AF20513) for Alzheimer's |
| | Disease: Prelude to a Clinical Trial |
| | Journal of Neuroscience 13 March 2013, |
| | 33 (11) 4923-4934; DOI: |
| | 12.2013 |
| UB 311 (United | Wang C et al. UB-311, a novel UBITh ® |
| Neuroscience) | amyloid β peptide vaccine for |
| | mild Alzheimer's disease. Alzheimers |
| | Dement (N Y). 2017 Apr 14;3(2):262-272. |
| | doi: 10.1016/j.trci.2017.03.005. |
| | eCollection 2017 Jun. |

An alternative active immunization approach is the DNA Aβ immunization in which not the peptide itself but a DNA encoding Aβ is injected. The injected DNA is translated in the immunized individual to produce Aβ peptide which then triggers respective immune responses against Aβ.

Thus in one embodiment, a vaccine or immune-stimulating for generating anti-Aβ antibodies may comprise a nucleic acid for encoding an immunogenic peptide for producing anti-Aβ antibodies, especially antibodies that bind to oligomeric Aβ42.

The nucleic acid may be DNA, in which case the vaccine is a DNA vaccine.

In one embodiment, the vaccine comprises a full length DNA encoding a Aβ trimer vaccine. The full length DNA Aβ trimer vaccine may contain B- and T-cell epitopes. The full-length DNA Aβ vaccine has the advantage that it is open to a wider anti-Aβ response with a broader variety of antibody epitopes.

DNA vaccines and use of same for generating or producing anti-Aβ antibodies are described in the documents referred to in Table 4. The entire contents of the documents referred to in Table 4 are incorporated herein by reference.

TABLE 4

| DNA vaccine for producing | |
|---|---|
| anti-Aβ42 antibodies | Reference |
| Aβ42 fused to an a-antitrypsin | Qu B, Rosenberg RN, Li L, Boyer |
| secretory signal upstream and | PJ, Johnston SA. Gene |
| a major histocompatibility | vaccination to bias the immune |
| complex II-targeting sequence | response to amyloid-beta |
| downstream | peptide as therapy for |
| | Alzheimer disease. Arch |
| | Neurol. 2004; 61:1859-1864. |
| pCA-PEDI-(Aβ1-6)11 | Kim HD, Jin JJ, Maxwell JA, |
| AdPEDI- (Aβ1-6) 11 | Fukuchi K. Enhancing Th2 |
| | immune responses against |
| | amyloid protein by a DNA prime- |
| | adenovirus boost regimen for |
| | Alzheimer's disease. Immunol |
| | Lett. 2007; 15:30-38. |
| Gal4/UAS DNA Aβ42 trimer | Qu BX, Lambracht-Washington D, |
| | Fu M, Eagar TN, Stüve O, |
| | Rosenberg RN. Analysis of |
| | three plasmid systems for use |
| | in DNA A beta 42 immunization |
| | as therapy for Alzheimer's |
| | disease. Vaccine. 2010; |
| | 28 (32):5280-5287. |
| pMDC-3Ab1-11-PADRE | Movsesyan N, Ghochikyan A, |
| | Mkrtichyan M, Petrushina 1, |
| | Davtyan H, Olkhanud PB, Head |
| | E, Biragyn A, Cribbs DH, |
| | Agadjanyan MG. Reducing AD- |
| | like pathology in 3xTg-AD |
| | mouse model by DNA epitope |
| | vaccine-a novel |
| | immunotherapeutic strategy. |
| | PloS One. 2008; 3:2124. |

### 3.6 Vaccine Administration

A vaccine or immune-stimulating composition described herein are administered by any appropriate standard routes of administration. The composition may be administered by topical, oral, rectal, nasal or parenteral (for example, intravenous, subcutaneous, or intramuscular) routes. In a particularly preferred embodiment, the composition or vaccine is administered by parenteral, particularly by i.p., i.v., s.c. or i.m injection.

A gene gun may be used for i.m or s.c injection using techniques known to the skilled worker.

The frequency of injection can be varied depending on the patient response. For example the frequency of administration can varied by the attending physician depending on the patient's response and corresponding antibody titers. For example, a patient who is a low responder may require more frequent administration, while a patient who is a high responder may require less frequent administration in order to elicit and/or maintain the same antibody titer.

The frequency of injection can include, but is not limited to, 1 to 10 administrations per year, e.g. 2 to 8 per year, e.g. 6 administrations per year.

In one embodiment, the composition or vaccine is administered to human patients in need thereof about every 4 to 8 weeks, preferably about every 5 to 7 weeks, in particular about every 6 weeks. Such a dosing regimen may last about 12 to 16 weeks, e.g. to about 12 weeks. For example, the composition or vaccine is administered at 0, 6, 12 weeks. Furthermore, the delay between subsequent administrations may be extended.

Thus, in one embodiment, the invention provides a dosing regimen of (a) two or more administrations at intervals of about 6 weeks, followed by (b) two or more administrations at intervals of about 12 weeks. In one embodiment, the invention provides a dosing regimen of (a) three administrations at intervals of about 6 weeks (e.g. at weeks 0, 6 and 12) followed by (b) two or more administrations (e.g. 3, 4, 5 or more) at intervals of about 12 weeks (e.g. at weeks 24, 36, 48 and 60).

According to the invention, about 5 to 600 μg of a peptide immunogen or DNA encoding same can be administered in human patients, for example about 5 to 550 g, about 50 to 500 μg, about 100 to 500 μg, e.g. about 75 to 300 μg, e.g. about 50 to 150 μg, e.g. about 15 to 125 μg, e.g. about 25 to 100 μg, e.g. about 50 μg, 75 μg, 100 μg, 150 μg, 200 μg, 300 μg, 400 μg or 450 μg. Thus, the composition or vaccine may contain one of these amounts per dose. In one embodiment, the composition of the invention comprises about 150 μg or about 50 μg of the peptide immunogen or DNA encoding same per dose.

### EXAMPLES

### Example 1—Materials & Methods

Aβ₄₂ administration study: Lyophilised recombinant Aβ₄₂ (Millipore) and scrambled control peptide (ScrAβ₄₂; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered 1 μg of recombinant Aβ₄₂ or ScrAβ₄₂ (n=10/group per cohort) by i.p. injection once/day for 5 wks. An i.p. glucose tolerance test (GTT) was performed on the final treatment day following an overnight fast. Mice were administered 2 g/kg lean mass of glucose including radioactive glucose tracers, prepared as follows. 100 μl of 1 μCi/μl glucose analogue, [³H]-2-deoxyglucose (2-DOG), and 500 μl of 200 μCi/mL U-¹⁴C glucose were evaporated to dryness before redissolving the radioactive tracers in 1 mL of 50% glucose. This produced a 50% glucose solution containing 100 μCi/mL [³H]-2-DOG and 100 μCi/mL U-¹⁴C glucose. The tail tip of each mouse was cut off and the blood glucose concentration of a blood sample was measured using an AccuCheck II glucometer (Roche). The GTT was initiated via intraperitoneal injection of the radiolabelled glucose solution (2 g/kg body weight, 10 uCi/animal) into the overnight-fasted mice. Further blood samples were taken at 15, 30, 45, 60 and 90 minutes after the injection for the measurement of blood glucose. Blood samples (30 μl) were also taken from the tail tip at each time point and diluted in 100 μl of saline. These samples were then centrifuged and the supernatant collected. 50 μl of the supernatant was diluted in 500 μl of distilled water and then suspended in 4 mL of Ultima Gold XR scintillation fluid (Packard Bioscience). Blood radioactivity was determined at each time point by performing liquid scintillation counting on each solution using the Beckman scintillation counter (LS6000 SC).

At the conclusion of the GTT, mice were killed via cervical dislocation. Blood was obtained immediately following by cardiac puncture and the heart, and other tissues were immediately removed. Hearts were washed in ice cold PBS and weighed prior to being snap frozen in liquid nitrogen. The heart (30 mg), epididymal fat pad (30 mg and quadriceps skeletal muscle (30 mg) were homogenised in 1.5 ml of distilled water. The homogenate was centrifuged at 3000 rpm for 10 min at 4° C. 400 μl of the supernatant was diluted into 1.6 mL of distilled water and then suspended in 14 mL of Ultima Gold XR scintillation fluid (Packard Bioscience). The radioactivity of each sample (from both [3H]-2-DOG6P and [³H]-2-DOG) was determined by liquid scintillation counting using the Beckman scintillation counter (LS6000 SC). The ³H radioactivity was used to measure glucose uptake into each tissue.

To determine the incorporation of U-¹⁴C glucose into triglyceride and the total triglyceride content in the heart, an extraction of triglyceride was carried out using a chloroform/methanol mixture. Samples of heart (30 mg) were hand-homogenised in 2 mL of chloroform/methanol (2:1) and the homogeniser rinsed in a further 2 mL of chloroform/methanol (2:1), and the washings being added to the original extract in 10 mL tubes. The tubes were tightly capped and mixed on a rotator overnight to maximise extraction of the triglycerides. 2 ml of 0.6% saline was then added, to facilitate the separation of the organic and aqueous phases, after which the tubes were mixed thoroughly and then centrifuged at 2000 rpm for 10 minutes. The lower chloroform phase (containing triglycerides) was collected and evaporated to dryness under nitrogen at 45° C. The dried extract was then re-dissolved in 250 μl of 100% ethanol, to redissolve the lipid and enable aliquots to dispensed for assay. The amount of U-¹⁴C glucose clearance into the lipid fraction was measured by suspending 100 μl of the triglyceride solution in 5 mL of Ultima Gold XR scintillation fluid (Packard Bioscience), followed by scintillation counting using the Beckman scintillation counter (LS6000 SC). Total triglyceride content was measured using an enzymatic fluorometric assay (BioVision) as per manufacturers' instructions. Lipoprotein lipase was used in an enzymatic reaction to yield fatty acid and glycerol. Quantified glycerol was used as an indirect measure of triglyceride and was 49ndividual to tissue weight.

Total mRNA from the tissues was extracted by homogenizing ˜20-30 milligrams of tissue in 1 ml of Trizol followed by incubation at room temperature (RT) for 5 min. 200 μL of chloroform was added to the homogenate, shaken for 15 seconds and incubated for 1 min at RT before centrifuging at 12,000 g for 10 min at 4° C. for extracting the upper aqueous phase. An equal volume (350 μl for cell lysate/450 μL for tissue) of 70% ethanol was added to cell/tissue samples and they were further purified with Rneasy spin columns (the Rneasy*min I Kit, Qiagen). Complementary DNA (cDNA) was synthesised using the SuperScript III transcription system (Invitrogen). cDNA was quantified by OliGreen assay (Quant-iT⁷™ OliGreen© ssDNA Assay Kit; Invitrogen). All primers were designed in-house using the Beacon Primer Designer program software and synthesised by Gene Works (Adelaide, Australia).

Primer sequence efficiency was tested over a wide concentration range. Gene expression levels were quantified using the FastStart Universal SYBR Green Master (ROX; Roche Applied-Science) on the MX3005P′ Multiplex Quantitative PCR (QPCR) system (Stratagene). Log-transformed CT values were normalised to cDNA concentration to determine relative gene expression levels.

The effect of Aβ₄₂ administration on cardiac function was assessed in another cohort of 12-week-old, male C57BL6 mice, which were administered Aβ₄₂ or ScrAβ₄₂ (n=10/group per cohort) by i.p. injection once/day for 5 wks. After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio.

Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05 [LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen.

3D6-High Fat Diet (HFD) prevention study: Male C57BL6 mice were obtained from the Animal Resource Centre (Perth, Wash.) at 4 weeks of age and housed 4 mice per cage on a 12 hr light/dark cycle at a temperature of 22° C. and a constant humidity with a normal rodent diet. At 12 weeks of age, echocardiography was performed on all mice (n=24), to obtain pre-treatment measures of cardiac function, as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s), as well as systolic measures such as ejection fraction and fractional shortening. An estimation of LV mass was calculated from the m-mode imaging by using the formula (1.05 [LVIDd+LVPWd+IVSd]³−[LVIDd]³) by Troy et al. (1972). All mice were then placed on a high fat diet (HFD) with 43% of calories from fat (23.5% by weight; SF04-001 High Fat Rodent Diet Based on D12451, Specialty Feeds, Glen Forrest, Wash.) for 13 weeks. At 12 weeks of age, mice were also administered 0.75 mg/kg bodyweight of either the Aβ₄₂ neutralising antibody 3D6 (#TAB-0809CLV, Creative Biolabs, Shirley, N.Y.) or the InVivo IgG2a Isotype Control antibody (#BE-0085, BioXCell, Lebanon, N.H.) weekly via intraperitoneal (i.p.) injection (n=12/group) for 13 weeks. Groups were selected based on fat mass, body weight and lean mass to match these variables as closely as possible between groups. Each cage contained 2 mice from each group.

After 10 weeks of the treatment period, mice underwent an oral glucose tolerance test (OGTT). Following a 5 hour fast, baseline readings of blood glucose were collected via a tail bleed of the mice using a hand-held glucometer (AccuCheck Performa). Mice were then administered 50 mg of glucose via oral gavage and blood glucose was measured 15, 30, 45, 60- and 90-minutes post administration. An additional 30 μL of blood was collected at baseline and 15, 30- and 60-minutes post administration in heparinised tubes for analysis of serum insulin concentration. Blood was centrifuged at 10,000 g for 10 minutes at 4° C. and plasma was collected by removing the supernatant. Plasma from the OGTT was analysed for insulin content using the Mouse Ultrasensitive Insulin ELISA (ALPCO, Salem, N.H.). An insulin tolerance test (ITT) 11 weeks into the treatment period. Following a 5 hour fast, baseline readings of blood glucose were collected via a tail bleed of mice using a hand-held glucometer (AccuCheck Performa). Mice were administered of 53ndivid via i.p. injection and blood glucose was measured 20, 40, 60, 90- and 120-minutes post administration. Echocardiography was then performed 12 weeks into the treatment period, as described above, to obtain post-treatment measures of cardiac function. Changes in cardiac function parameters were expressed as a percentage of the baseline measure. Mice were sacrificed following 13 weeks of the treatment period. At the conclusion of the treatment period, mice were killed via cervical dislocation following a 5-hr fasting period. Blood was obtained immediately following by cardiac puncture and the heart, and other tissues were immediately removed. Hearts were washed in ice cold PBS and weighed prior to being snap frozen in liquid nitrogen.

3D6-High Fat Diet (HFD) treatment study: At 12 weeks of age, echocardiography was performed on mice (n=36) to obtain baseline measures of cardiac function. Mice were then separated into 3 groups of 12, which included a chow/control, HFD/control and HFD/3D6 group. The groups were selected based on their measures of diastolic function, fat mass and bodyweight, to match these variables as closely as possible. The two HFD groups were then placed on a HFD with 43% of calories from fat (23.5% by weight; SF04-001 High Fat Rodent Diet Based on D12451, Specialty Feeds, Glen Forrest, Wash.) for 22 weeks, while the chow group remained on a standard chow diet. Following 15 weeks of the diet period, echocardiography was again performed on all groups to obtain pre-drug treatment measures of cardiac function. The chow/control and HFD/control groups were then administered 0.75 mg/kg bodyweight of the InVivo IgG2a Isotype Control antibody (#BE-0085, BioXCell, Lebanon, N.H.) weekly via I.P injection for 7 weeks while the HFD/3D6 group received 0.75 mg/kg bodyweight of the 3D6 antibody (#TAB-0809CLV, Creative Biolabs, Shirley, N.Y.). Echocardiography was then performed following 6 weeks of the treatment period to obtain post-drug treatment measures of cardiac function. Following 7 weeks of the drug administration, mice were humanely killed via cervical dislocation and blood was immediately obtained via cardiac puncture and stored in a heparinised tube. The heart, epididymal fat pad, mesenteric fat pad, liver, quadricep, hind limb and brain were then immediately dissected. The heart was 151 blotted prior to being weighed and all tissues were snap frozen in liquid nitrogen and stored at−80° C. Plasma Aβ42 was measured using a high sensitivity ELISA kit (Wako Diagnostics) and plasma that was diluted 1:10 with assay buffer. Cardiac TAG was measured using using a triglyceride GPO-PAP kit (Roche Diagnostics) after extraction by KOH hydrolysis.

Aβ₄₀ administration study: Lyophilised recombinant Aβ₄₀ (Millipore) and scrambled control peptide (ScrAβ₄₀; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI.

Mice were then administered 1 μg of recombinant Aβ₄₀ or ScrAβ₀₂ (n=12/group per cohort) by i.p. injection once/day for 5 wks.

After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05 [LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen. Plasma Aβ40 was measured using a high sensitivity ELISA kit (Wako Diagnostics) and plasma that was diluted 1:10 with assay buffer.

### Example 2—Chronic Aβ42 Administration Alters Cardiac Metabolism

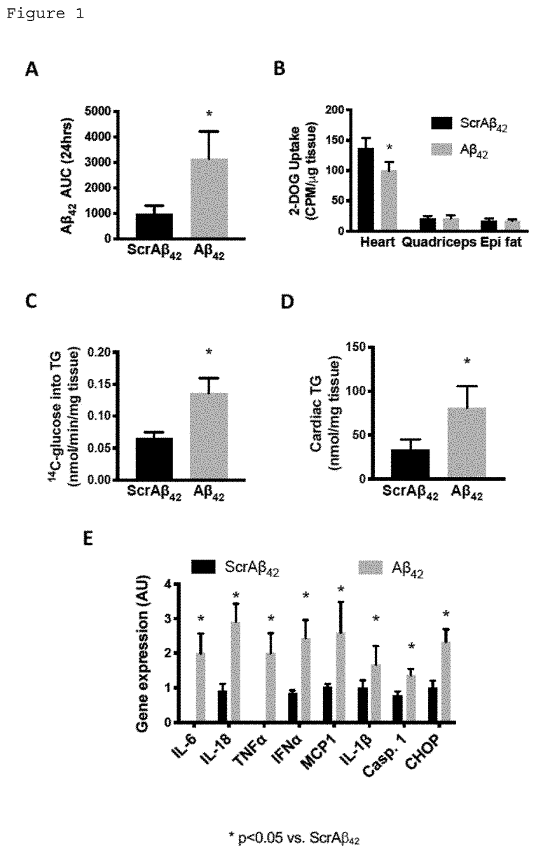

The in vivo effects of Aβ₄₂ were assessed by i.p. administration of 1 μg/day of Aβ₄₂, while control mice were administered a scrambled Aβ₄₂ peptide (ScrAβ₄₂) for a period of five weeks. Administration of Aβ₄₂ increased plasma Aβ₄₂ approximately 3-fold compared with administration of ScrAβ₄₂ (FIG. 1A) There was no change in body weight, body composition or food intake in mice administered Aβ₄₂. After five weeks of peptide administration, a GTT with glucose tracers was performed. There was no difference in whole body glucose tolerance or plasma insulin throughout the GTT between ScrAβ₄₄ or Aβ₄₂ administered mice. However, when tissues were assessed for glucose uptake throughout the GTT, by 2-DOG uptake, an ˜25% decrease in glucose uptake by the heart was observed in mice administered Aβ₄₂ (FIG. 1B). Glucose utilisation was further analysed using ¹⁴C-glucose labelling which revealed greater glucose incorporation into TAG (FIG. 1C) and increased total TAG (FIG. 1D) in Aβ₄₂ administered mice. This was associated with gene expression changes indicative of cardiac stress responses, including inflammation and endoplasmic reticulum stress (FIG. 1E).

### Example 3—Chronic Aβ42 Administration Alters Cardiac Function

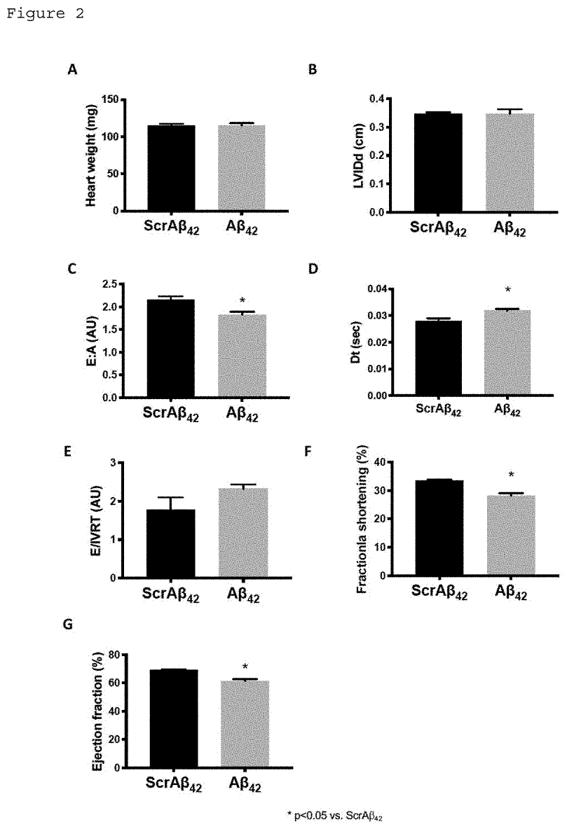

To assess whether Aβ₄₂ administration affected cardiac function, mice were administered ScrAβ₄₂ or Aβ₄₂ for five weeks prior to echocardiography. Hearts were also collected for morphological analysis (FIG. 2). Administration of Aβ₄₂ had no effect on gross heart weight (FIG. 2A) or of internal dimensions of the left ventricle (LVIDd; FIG. 2B). However, indices of diastolic dysfunction were evident in mice administered Aβ₄₂, including reduced E:A ratio (FIG. 2C) and increased deceleration time (FIG. 2D). There was no significant difference between groups when the peak blood flow velocity into the left ventricle during the relaxation phase in early dystole I was normalised by the relaxation time (isovolumetric relaxation time; IVRT) (FIG. 2E). This is a index of left artrial pressure and suggests that the diastolic dysfunction observed could be classified as grade 1. Furthermore, fractional shortening (FIG. 2F) and ejection fraction (FIG. 2G) were both reduced in Aβ₄₂ administered mice, which is indicative of systolic dysfunction.

### Example 4—Administration of Anti-Aβ42 Antibodies Preserves Diastolic Function in Development of Obesity

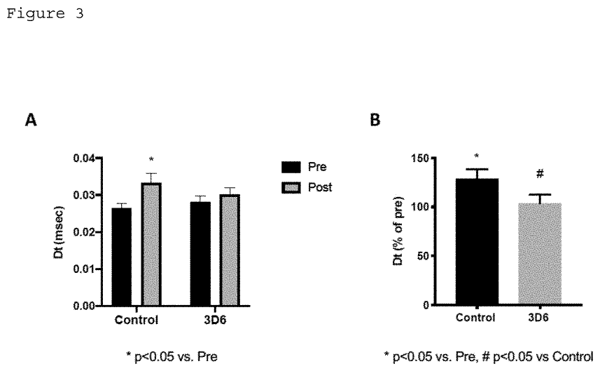

Echocardiography Doppler imaging of the mitral valve was used to assess the deceleration time, a critical measure of diastolic function (FIG. 3). Following 14 weeks of high fat feeding, mice administered the control antibody had an increase in deceleration time (FIG. 3A), indicating deterioration of diastolic function. In contrast, mice administered the 3D6 antibody showed either preserved or decreased deceleration time (FIG. 3A). Expressed relative to baseline measures, mice administered the control antibody had a statistically significant ˜30% increase in deceleration time (FIG. 3B), indicative of diastolic dysfunction. In contrast, deceleration time in mice administered the 3D6 antibody did not change from baseline levels (FIG. 3B). The relative change in deceleration time from baseline was significantly different between control and 3D6 antibody administered groups (FIG. 3B).

### Example 5—Administration of Anti-Aβ42 Antibodies Prevents Concentric Hypertrophy in Development of Obesity

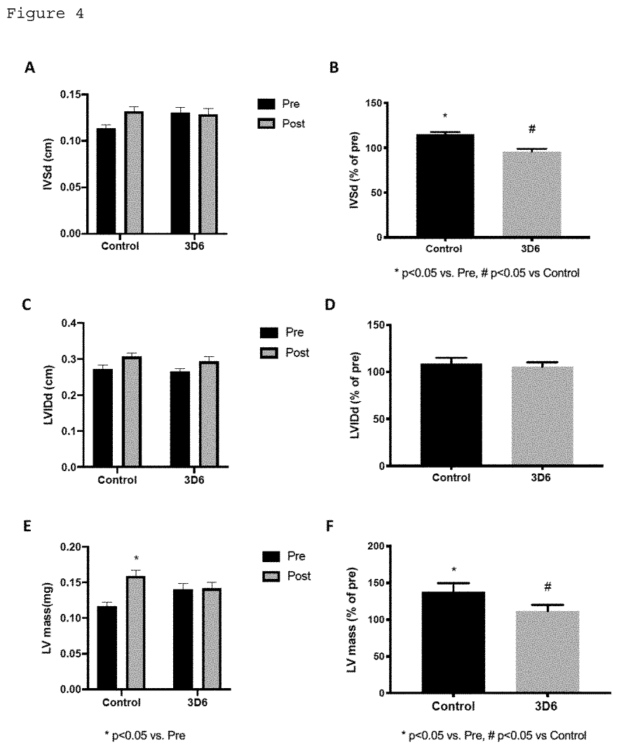

Echocardiographic M-mode imaging was used to characterise the morphology of the left ventricle (FIG. 4). Mice administered control antibody tended to have an increased intraventricular septum thickness at end-diastole (IVSd), a measure of hypertrophy, following the development of obesity, which was not observed in mice administered 3D6 antibody (FIG. 4A).

Expressed relative to pre high fat diet values, IVSd significantly increased 115% in mice administered control antibody, while in mice administered 3D6 antibody this value was 95% (FIG. 4B). The relative change in IVSd from pre high fat diet values was significantly different between control and 3D6 antibody administered groups (FIG. 4B). There were no differences between the left ventricle internal diameter at end-diastole (LVIDd), a measure of left ventricle dilation, between groups (FIGS. 4C and D). However, mice administered control antibody significantly increased calculated left ventricular mass, a measure of hypertrophy, throughout the development of obesity, which was not observed in mice administered 3D6 antibody (FIG. 4E). Expressed relative to pre high fat diet values, left ventricular mass significantly increased 138% in mice administered control antibody (FIG. 4F). The relative change in left ventricular mass from pre high fat diet values was significantly different between control and 3D6 antibody administered groups (FIG. 4F).

### Example 6—Administration of Anti-Aβ42 Antibodies Preserves Diastolic Function and Reduces Cardiac TAGs in Established Obesity

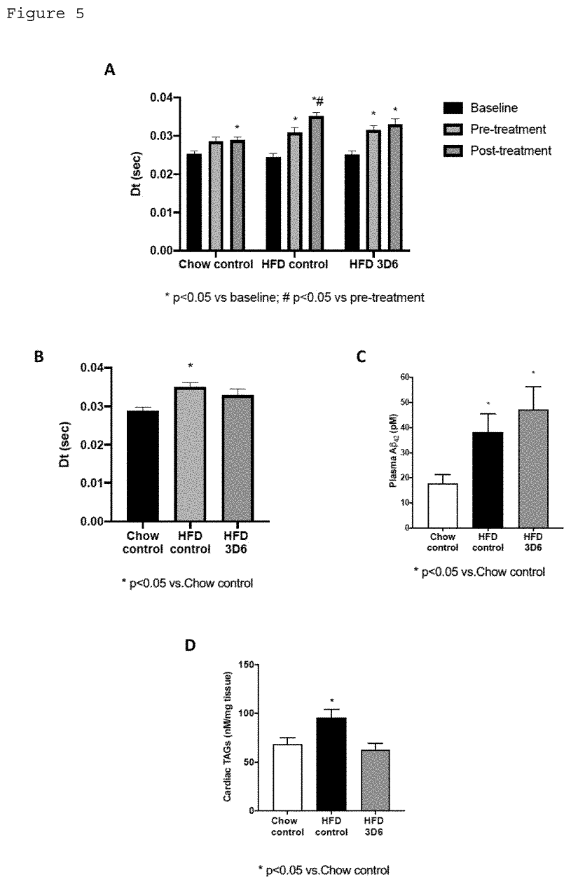

To assess the effect of treating obese mice with 3D6 on diastolic function, Doppler imaging of the mitral valve was conducted using echocardiography at the start of the study (Baseline), after 13 weeks of chow or HFD (Pre-treatment) and following 7 weeks of weekly 3D6 administration (Post-treatment) (FIG. 5).

In the chow control group, there was no significant change in DT between Baseline and Pre-treatment, but DT was significantly increased at Post-treatment compared with Baseline (FIG. 5A).

In the HFD control group, DT significantly increased from Baseline to Pre-treatment and was further increased at Post-treatment (FIG. 5A). In contrast, the HFD 3D6 group showed a significant increase in DT between Baseline and Pre-treatment, however diastolic function did not deteriorate any further following 3D6 administration (FIG. 5A). When examining DT between groups at the conclusion of treatment period, DT was significantly elevated in the HFD control group compared to the Chow control group, while DT was not significantly different from Chow control in the HFD 3D6 group (FIG. 5B). The effect of the intervention on plasma Aβ42 was examined. In the HFD control group, Aβ42 levels were significantly increased compared with the Chow control group (FIG. 5C). Consistent with the neutralising function of the 3D6 antibody, plasma Aβ42 remained elevated in the HFD 3D6 group compared with Chow control (FIG. 5C). However, 3D6 treatment reduced cardiac TAG accumulation in obese mice (FIG. 5D).

### Example 7—Aβ40 Chronic Administration does not Alter Cardiac Function

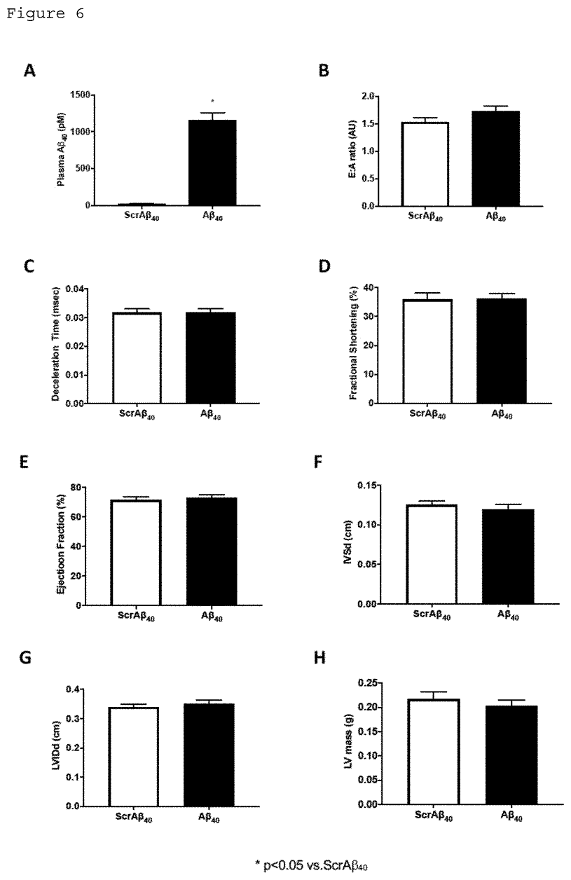

To determine whether other amyloid beta peptides could induce cardiac dysfunction similar to Aβ42, mice were administered Aβ40 or scrambled Aβ40 (ScrAβ40) at 1 μg/day by i.p. injection for 5 weeks, prior to echocardiography (FIG. 6). Administration of Aβ40 significantly increased plasma Aβ40 (FIG. 6A). However, administration of Aβ40 did not have any effect on indices of diastolic function, including E:A ratio (FIG. 6B) and DT (FIG. 6C), nor any effect on indices of systolic function, including fractional shortening (FIG. 6D) and ejection fraction (FIG. 6E). In addition, Aβ40 administration had no effect on cardiac morphology measures, including IVSd (FIG. 6F), LVIDd (FIG. 6G) and LV mass (FIG. 6H).

### Example 8—Discussion and Conclusion

These data indicate that Aβ42 alters cardiac metabolism and function and has particular impact on diastole. Without being bound by hypothesis, it is believed that the alteration or reprogramming of cardiac metabolism may arise from an Aβ42 mediated or associated inflammatory response.

Administration of Aβ42 to mice reduced cardiac glucose uptake and shunted glucose into TAG synthesis, leading to TAG accumulation. Reduced glucose uptake and utilisation increases the reliance on fatty acid oxidation, which reduces cardiac efficiency. This is due to the greater O₂ cost to produce ATP from beta oxidation, which impairs ATP production and results in impaired cardiac relaxation. This leads to impaired diastolic function because the diastolic relaxation phase has large energetic and ATP requirements, as Ca2+ reuptake and normalisation of membrane ion balances is ATP dependent. Further, the relaxation phase is much longer than systole. Hence the increased reliance on fatty acid oxidation leads to the observed diastolic dysfunction.

Reduced glucose uptake and TAG accumulation are phenotypic traits of cardiomyopathy associated with obesity, whereby altered cardiac metabolism leads to impaired relaxation of the heart, or diastolic dysfunction, which is sufficient to initiate progression to heart failure. Over time, this can lead to concentric hypertrophy and can often also present with impaired systolic function. Consistent with this, administration of Aβ42 to mice impaired both diastolic and systolic function. However, these effects on cardiac function were not observed in mice administered Aβ40, suggesting that the effects of amyloid $on the heart are restricted to the isoform of 42 amino acids.

These data also indicate that inhibiting Aβ42 function can prevent the development of diastolic dysfunction in obesity and in other individuals having a higher than normal plasma amount of Aβ42 and protein comprising same. Administration of the 3D6 Aβ42 neutralising antibody to mice throughout high fat feeding prevented the decline in diastolic function and development of concentric hypertrophy, represented by changes in IVSd and left ventricle mass, without left ventricle dilation (LVIDd).

Furthermore, administration of the 3D6 Aβ42 neutralising antibody to mice with established obesity-induced diastolic dysfunction prevented further declines in diastolic function and reduced cardiac TAG accumulation.

These data therefore indicate that Aβ42 antibodies could be used to prevent diastolic dysfunction and halt progression to heart failure in obesity and conditions in individuals having a higher than normal plasma amount of Aβ42.

### Example 9

Vaccination to generate Aβ42 antibodies is utilised to prevent obesity-associated cardiomyopathy or cardiomyopathy where circulating Aβ42 is elevated. CAD106 may be administered in a single administration of the immunizing peptide in PBS at doses of 2.5-250 mg/kg via subcutaneous injection. The vaccination protocol could also include additional intramuscular administrations of the immunizing peptide 1, 2 and 3 months after the initial immunization.

1. A method for preventing or treating diastolic dysfunction in an individual comprising providing in an individual in need of said prevention or treatment a therapeutically effective amount of an anti-Aβ antibody.

2. The method of claim 1 wherein the anti-Aβ antibody is Bapineuzumab.

3. The method of claim 2 wherein the individual has diastolic dysfunction.

4. The method of claim 3 wherein the individual has decreased cardiac glucose uptake.

5. The method of claim 4 wherein the individual has increased glucose incorporation into triacyl glycerol (TAG) in cardiac tissue.

6. The method of claim 5 wherein the individual has increased total cardiac TAG.

7. The method of claim 1 wherein the individual has a reduced E:A ratio.

8. The method of claim 1 wherein the individual has an increased deceleration time.

9. The method of claim 1 wherein the individual has increased intra ventricular septal thickening.

10. The method of claim 1 wherein the individual has increased left ventricle (LV) mass.

11. The method of claim 1 wherein the individual has an elevated plasma amount of A42.

12. The method ofany one of the claim 1 wherein the anti-Aβ antibody preserves or decreases E wave deceleration, thereby minimising diastolic dysfunction.

13. The method of claim 1 wherein the anti-Aβ antibody prevents concentric hypertrophy.

14. The method of claim 1 wherein the anti-Aβ antibody preserves or prevents intra-ventricular septal thickening.

15. The method of claim 1 wherein the anti-Aβ antibody preserves LV mass or prevents increased LV mass.

16. The method of claim 1 wherein the individual is obese.

17. The method of any one of claim 1 wherein the individual is pre-diabetic, or diabetic.

18. The method of claim 1 wherein the individual does not have Alzheimer's disease.

19. The method of claim 1 comprising administering an anti-Aβ antibody to the individual to provide in an individual a therapeutically effective amount of an anti-Aβ antibody.

20. The method of claim 1 comprising administering Aβ to the individual to provide in an individual a therapeutically effective amount of an anti-Aβ antibody.

21. The method of claim 1 wherein the individual has been assessed to determine whether the individual has an elevated amount in plasma of Aβ42.

22. The method of claim 21 wherein the individual is provided with a therapeutically effective amount of anti-Aβ antibody, or wherein the individual is provided with an Aβ peptide to provide in the individual a therapeutically effective amount of anti-Aβ antibody, where the individual has been assessed as having an elevated plasma amount of Aβ42 peptide.
